(19) United States
(12) Patent Application Publication
(10) Pub. No.: US 2011/0218552 A1
(43) Pub. Date: Sep. 8, 2011

(54) System and method of removing skin lesions

(76) Inventors: Roger P. Hoffman, Punta Gorda, FL (US); David Bertler, Hobart, WI (US)

(21) Appl. No.: 12/660,809

(22) Filed: Mar. 4, 2010

(51) Int. Cl. A61B 17/50

(52) U.S. Cl. 606/131

(57) ABSTRACT

A system and method for the removal of various skin lesions located on or in the outer layer of the skin.

### FIELD OF THE INVENTION

The present invention relates to a system and method for the removal of various skin lesions located on the outer layer of the skin.

### BACKGROUND

There are both benign and malignant conditions that can occur on the epidermis. These range from various types of skin cancers to benign warts, skin tags, birth marks, scars and wrinkles. There are various types of treatments for these conditions that range from surgical, chemical and light-based interventions as well as compounded salves and ointments. While the efficacy of these treatments vary it is clear that there is a need for additional and more effective treatments.

U.S. Pat. No. 6,447,463 relates to a hand portable diagnostic kit. The kit is used for transferring one or more skin lesions to a remote location such as a diagnostic lab. The kit can include a plastic diagnostic slide with attached self-adhesive tape, a plastic specimen bag with which the slide with attached skin lesion can be placed. Also, included is medical information, use instructions and a return envelope.

U.S. Pat. No. 7,476,205 relates to an apparatus for treatment of patients who have lesions distributed on the surface of their skin. A body cover has one or more cover elements, the cover elements having a gas type layer and being flexible to fit to body parts in a tight manner. In the areas where there are skin lesions, there are provided open-pored sponge-like bodies on the side of the gas tight layer facing the patients body as well as connection ports for the exchange of fluids.

U.S. Pat. No. 4,112,121 relates to a method and composition for the treatment of seborheic keratosis. The composition consists essentially of an alakine metal or an ammonium phenate, a monohydric alcohol and polyhydric alcohol humectant and a gelling agent.

U.S. Patent Publication No. 20030087960 relates to a pharmaceutical preparation for treating a skin or mucus membrane lesion. The pharmaceutical preparation includes trichloloracetic acid and hydrochloric acid, or trichioroacetic acid and formic acid or all three of these acids and optionally a cross-linking/fixating/preserving agent.

### SUMMARY OF THE INVENTION

This present invention relates to the removal of the various lesions by utilizing the fact that the lesion is typically on or in the outer layer of the skin. In addition the lesion is by definition different than the surrounding tissue—hence it is a lesion. The lesion has a greater affinity for the tissue that it is constituted of than for the tissue to which it is attached.

The present invention relates to selectively applying a force to the lesion and removing it based on the difference in the bonding strength within the differing tissue than the bonding of the differing tissue to the normal tissue that surrounds it.

It is an object of the present invention to increase the efficacy of this treatment, by first treating the lesion with a pretreatment that could be either a chemical or an enzymatic treatment.

It is an object of the present invention to remove the lesion by use of an adhesive tape. It is an object of the present invention for the aggressiveness or tack of the tape to be calibrated by testing on a similar area of skin in a non-lesional containing area. This way the physician can ensure that the tape does not permanently damage the healthy epidermis. It is a further object of the invention to paint the lesion with an adhesive. In this way, non-lesional tissue experience minimal or no applied force.

It is an object of the present invention for the lesion to be painted with an adhesive that adheres to the lesion and then dries so that the exposed area is not tacky once the adhesive is dry. Once dried, the material while not tacky to the touch, bonds to a similar material that is also dried on contact. The advantage of these types of applications is that in this manner there is no force applied to the normal surrounding area.

It is an object of the present invention for the affected lesional area to first be pretreated with a chemical or enzyme. This reduces the internal bonding within the defect, and also reduces the bonding strength to the surrounding tissue. An advantage of the present invention is that the treatment is very mild.

It is an object of the present invention for the physician after he first inspects the skin and ensures that this method of treatment is appropriate, he would apply the pretreatment. The patient then waits for the pretreatment to work, or applies additional pretreatment material in a non-clinical setting. When the lesion has been adequately treated, the patient or the physician removes the lesion by applying the adhesive tape.

There are many possible variations to this procedure that could include the use of this adhesive approach on a continual basis with perhaps intervening time being allowed for the treated lesional area to heal. It is an object of the invention to remove the lesion in layers.

It is an object of the invention for the types of lesion to range from various types of skin cancers, warts, skin tags to birth marks, scars and wrinkles.

It is an object of the present invention for the system and method to remove such entities as wrinkles, scars or birthmarks in this fashion. The treatment—pretreatment chemical/enzyme and the adhesive is non-aggressive enough that the patient is comfortable performing these actions without the need to see a physician other than at the beginning of the treatment to ensure that the procedure was appropriate, and only then for follow-up in the event that there were some complications that resulted from the treatment.

It is an object of the present invention for a lesion to have different properties such as porosity and reaction with fluids, wettability. For example, a lesion may be more hydrophilic or have a different density than the area around it. Therefore it is an object of the invention for a pretreatment to be designed in a way to react more quickly with the lesion than the healthy dermis. For example, a skin cancer could be treated if it was more porous to allow it to be penetrated more rapidly than the surrounding tissue.

It is an object of the present invention for a pretreatment to soften the lesion.

It is an object of the present invention for glacial acetic acid to be used to soften a lesion and therefore change the bonding between the lesion and the surrounding tissue.

It is an object of the present invention to increase the internal strength of the legion so that it can be pulled out of the healthy tissue completely.

It is an object of the present invention to treat healthy tissue around the lesion with a lubricant so that an adhesive does not adhere to the healthy tissue.

### DETAILED DESCRIPTION OF THE INVENTION

The present invention relates to the removal of various skin lesions by going after the fact that the lesion is typically on or in the outer layer of the skin. The lesion is by definition different than the surrounding tissue—hence it is a lesion. The lesion has a greater affinity for the tissue that it is constituted of than for the tissue to which it is attached, therefore, one can selectively apply a force to the defect and remove it based on the difference in the bonding strength within the lesion tissue than the bonding of the lesion tissue to the normal tissue that surrounds it.

In an embodiment, to further increase the efficacy of the treatment, the lesion is first treated with a pretreatment that could be either a chemical or an enzymatic treatment in nature.

In an embodiment, the separation is accomplished with an adhesive tape. In this case the aggressiveness or tack of the tape is calibrated by testing on a similar area of skin in a non-affected area. This way the physician can ensure that the tape does not damage the healthy epidermis.

In a further embodiment, the lesion is “painted” with an adhesive. In this way normal tissue experiences minimal or no applied force. In another embodiment the lesion is painted with an adhesive that adheres to the lesion and then dried so that the exposed area is not tacky once the adhesive is dry. Once dried, the material while not tacky to the touch, bonds to a similar material that is also dried on contact. The advantage of these types of embodiments is that in this manner there is minimal force applied to the normal surrounding area.

In an embodiment, the lesion site area is first pretreated with a chemical or enzyme. This reduces the internal bonding within the lesion, and also reduces the bonding strength to the surrounding tissue. One advantage of this invention is that the treatment is very mild. After the physician first inspects the lesion and ensures that this method of treatment is appropriate, he applies the pretreatment. The patient would allow time for this pretreatment to work, or could even apply additional pretreatment material in a non-clinical setting. When the lesion has been adequately treated the patient or the physician removes the lesion by applying the adhesive tape.

There are many possible variations to this procedure that could include the use of this adhesive approach on a continual basis with perhaps intervening time being allowed for the prior treated area to heal. In this case it is possible to remove the lesion in layers. In an embodiment, the procedure of the present invention removes such varied conditions as wrinkles, scars or birth marks in this fashion. The treatment—pretreatment chemical/enzyme and the adhesive—is non-aggressive enough that the patient is comfortable performing these actions without a repeat physician visit. A physician visit would be needed at the beginning of the treatment to ensure that the procedure was appropriate, and only then for follow-up in the event that there were some complications that resulted from the treatment.

1. A method for removing various skin lesions comprising:
selectively applying a force to said lesion; and
removing said lesion based on difference in bonding strength within said lesion tissue than bonding of said lesion tissue to normal tissue that surrounds it.

2. The method of claim 1 wherein said lesion is located on or in outer layer of said skin.

3. The method of claim 1 wherein said lesion has a greater affinity for tissue that it is constituted of than for tissue to which it is attached.

4. The method of claim 1 further comprising:
treating said lesion with a pretreatment of either a chemical or an enzymatic treatment.

5. The method of claim 1 wherein said force is applied by use of an adhesive tape.

6. The method of claim 5 further comprising:
calibrating tack or aggressiveness of said tape by testing on a similar area of skin in a non-lesional containing area.

7. The method of claim 6 wherein said tape does not damage healthy epidermis.

8. The method of claim 1 further comprising using an adhesive on a continual basis with intervening time allowed for defective area to heal.

9. The method of claim 1 further comprising removing said lesion in layers.

10. A method of removing various skin lesions comprising:
painting an adhesive to said lesion;
drying said adhesive so that exposed area is not tacky once said adhesive is dry;
bonding said lesion to a similar material that is also dried on contact.

11. The method of claim 1 further comprising:
determining properties of lesion including porosity and reaction with fluids, wettability.

12. The method of claim 11 further comprising:
pretreating said lesion based on properties of said lesion.

13. The method of claim 12 wherein said pretreatment softens said lesion.

14. The method of claim 13 wherein glacial acetic acid is used to soften said lesion.

15. The method of claim 1 further comprising:
increasing internal strength of said lesion so that it can be pulled out of healthy tissue completely.

16. The method of claim 5 further comprising:
treating healthy tissue around said lesion with a lubricant so that said adhesive does not adhere to it.
